(19) United States
(12) Patent Application Publication
(10) Pub. No.: US 2010/0297055 A1
(43) Pub. Date: Nov. 25, 2010

(54) COSMETIC COMPOSITION COMPRISING MULTIFUNCTIONALISED POLY (ALPHA OLEFIN-COPOLYMER-MALEIC ANHYDRIDE)

(75) Inventors: Pietro Rando, Opera (IT); Giuseppe Maio, Zelo Surrigone (IT); Dario Magni, Bernareggio (IT)

Correspondence Address: SEED INTELLECTUAL PROPERTY LAW GROUP PLLC, 701 FIFTH AVE, SUITE 5400, SEATTLE, WA, 98104, US

(73) Assignee: INTERCOS S.P.A., Milano (IT)

(21) Appl. No.: 12/440,998

(22) PCT Filed: Sep. 12, 2006
(86) PCT No.: PCT/IT06/00661

§ 371 Date: Oct. 29, 2009

(51) Int. Cl. A61K 8/72; B05D 1/02; A61Q 19/00

(52) U.S. Cl. 424/78.03; 427/2.1

(57) ABSTRACT

The present invention relates to cosmetic compositions comprising multiple functionalized maleic anhydride/α-olefin copolymers which have been functionalized with a first reactive —OH or —NH₂ group containing organic compound and subsequently with a second reactive —OH or —NH₂ group containing organic compound, different from the first one. Typical examples are multiple functionalized copolymers in which the first reactive —OH group containing compound is a C₁₄-C₂₈ monoalcohol, or a sitosterol, and the second reactive —OH group containing compound is bis-hydroxypropoxyethyldimethicone or bis-hydroxyethoxypropyldimethicone. The cosmetic compositions are particularly suitable for application to the skin, the lips and keratinous materials.

### BACKGROUND OF THE INVENTION

1. Field of the Invention

The present invention relates to cosmetic compositions comprising multiple functionalized polyanhydrides. More particularly, it relates to cosmetic compositions comprising multiple functionalized maleic anhydride/α-olefin copolymers.

2. Description of the Prior Art

Maleic anhydride/α-olefin copolymers are well-known in the art. They have been proposed for numerous applications in the cosmetic field. Derivatives of these copolymers have also been described in the art. Thus, for example, U.S. Pat. No. 2,615,845 (Lippincott et al.) describes esters of these copolymers with straight-chain C₁₀-C₁₈ alcohols as lubricating oil additives. Similarly, U.S. Pat. No. 4,151,069 (Rossi) describes esters of maleic anhydride/C₁₈-C₅₀ α-olefin copolymers with C₁₈-C₃₀ linear alcohols as dewaxing aids for lubricating oils. In U.S. Pat. No. 4,548,725 (Bridger) C₄-C₄₀ alkyl esters of maleic anhydride/α-olefin copolymers are described for lowering the cloud point of a mineral oil.

All of these prior references refer to half—or complete alkyl esters of the copolymer, i.e. the derivative contains only an alkyl group as functional group.

From a technical leaflet of Chevron Phillips Chemical Company LLC of 2005 concerning Polyanhydride Resins a product PA-18 is known which is a polyanhydride resin derived from 1-octadecene and maleic anhydride. This product and its derivatives are stated to be useful in several applications, such as thickening agents, dispersing agents and as water proofer for personal care products. PA-18 can according to this leaflet react with inter alia amines to form amides or imides, and with alcohols to form half-esters or diesters.

From U.S. Pat. No. 6,492,455 (Nadolsky) it is Known, that reaction products of C₆ and higher α-olefin/maleic anhydride copolymers and polyfunctionalized amines can be used in cosmetic applications such as hair sprays. In U.S. Pat. No. 6,025,501 (Ulmer, et al.) alkyl half-esters of maleic anhydride/α-olefin copolymers, optionally with repeat maleamic acid units or maleimide are described for use in personal care products such as hair sprays. In U.S. Pat. No. 6,423,785 (Esselborn, et al.) maleic anhydride copolymers with functional amine oxide groups are described as dispersants for pigments. Maleic anhydride copolymers which have been cross-linked with a cross linking agent containing at least two cross linkable groups have been described in U.S. Pat. No. 6,706,817 (Plochocka) for use as bioadhesive hydrogels in inter alia cosmetic delivery systems.

However, in so far as the above prior art describes functionalized derivatives of maleic anhydride/α-olefin copolymers for use in cosmetic applications, they are single functionalized derivatives, that is to say that they contain one or more of the same functional groups. In contrast thereto, the functionalized maleic anhydride/α-olefin copolymers of the present invention are multiple functionalized, that is to say that they contain at least two different functional groups. According to the present invention it has been found that reacting a maleic anhydride/α-olefin copolymer with a first reactive —OH or —NH₂ group containing organic functionalizing compound to open part of the anhydride rings and treating the resulting modified copolymer with a second reactive —OH or —NH₂ group containing organic functionalizing compound to open all or nearly all of the remaining anhydride rings, said second reactive compound being different from the first reactive compound, produces a multiple functionalized copolymer with unexpected cosmetic benefit properties as will be described in more detail hereinafter. “Reactive” means tint the hydroxy- or amine-group is capable of ring-opening of the anhydride and reacting with a carboxy-group of the anhydride after ring-opening of the anhydride. “Different” means not belonging to the same chemical family.

### SUMMARY OF THE INVENTION

Thus, in its broadest aspects, the present invention relates to cosmetic compositions comprising a multiple functionalized maleic anhydride/α-olefin copolymer which has been multiple functionalized by reacting the maleic anhydride/α-olefin copolymer with a first reactive —OH or —NH₂ group containing organic functionalizing compound and subsequently with a second, different reactive —OH or —NH₂ group containing organic functionalizing compound.

### DETAILED DESCRIPTION OF THE INVENTION

The maleic anhydride/α-olefin copolymers, used in the present invention are those, well-known in the art. The α-olefins are C₈-C₅₀, preferably C₁₀-C₂₄ and particularly preferably C₁₈ α-olefins. They are usually prepared by reacting approximately equimolar amounts of maleic anhydride and α-olefin in the presence of a free radical initiator. The number of repeat units in the copolymer may vary from 2 to 100, and the number average molecular weight may vary from about 350 to about 600,000. A preferred maleic anhydride/α-olefin copolymer is a maleic anhydride/1-octadecene copolymer with a molecular weight in the range of 20.000 to 50.000.

For brevity's sake, the starting maleic anhydride/α-olefin copolymer will hereinafter be referred to as MA/O copolymer.

The multiple functionalized MA/O copolymer of the present invention are prepared by reacting the copolymer with a first reactive hydroxy—or amine group containing organic functionalizing compound to open part of the anhydride rings, and subsequently with a second, different reactive hydroxy—or amine group containing organic functionalizing compound to open all or nearly all of the remaining anhydride rings. A great variety of such compounds can be used as first or second reactive compound, such as ‘skin active’ compounds such as ceramides, panthenylethyl ether, sterols such as sitosterols, tocopherols; linear/branched saturated/unsaturated C₂-C₃₄ alcohols; alkyl amines, alkylhydroxy esters, sorbitol esters, sucrose esters; hydrophilic compounds such as glycerol, its mono and di-esters, polyglycerol, amino acids and their derivatives, mono and polysaccharides, ethoxylated alcohols; fluorinated oligomers and polymers; natural compounds capable of UV screening such as tannins, flavonoids, thymol, caffeic acid esters and vitamin E. Other suitable compounds can be selected from cosmetic benefit agents, skin-conditioning agents, vitamins, anti-acne-actives, anti-wrinkle agents, sunscreen actives and so on. Care should be taken, of course, that the first and the second reactive compound are different. Mixtures of the above compounds can, of course, also be used as the first or the second reactive compound, as long as the mixture of first reactive compounds is different from the mixture of the second reactive compounds. Preferred reactive —OH group containing compounds are straight chain alcohols with 8-30, preferably 14-28 carbon atoms. Another preferred group of reactive —OH group containing compounds are the sit sterols. Yet another preferred group of reactive —OH group containing compounds are hydroxy-terminated silicones such as the siliconols. Examples of the latter are bis-hydroxyethoxypropyldimethicone and bis-hydroxypropoxyethyldimethicone. Preferred reactive —NH₂ group containing compounds are primary amines. A typical example of a multiple functionalized MA/O copolymer of the invention is one wherein the first reactive compound is a C₁₄ or a C₂₈ straight chain alcohol and the second reactive compound is a siliconol as exemplified above.

The reaction of the MA/O copolymer with the first reactive hydroxy—or amine group containing organic compound to open part of the anhydride rings can be carried out in a manner, known per se, e.g. as described in U.S. Pat. No. 3,531,440 or U.S. Pat. No. 2,615,845. By varying the reaction temperature, reaction time, catalyst and relative molar amounts of reactants the number of opened rings and functionalizing groups formed can be controlled. In general, from 5 to 90%, usually from 20% to 80%, preferably from 30 to 70%, and particularly preferably 50% of the available anhydride rings should be opened in this way The resulting reaction product, which contains, apart from the functionalizing groups, still unopened anhydride rings is hereinafter for brevity's sake referred to as “modified MA/O copolymer”

The thus obtained modified MA/O copolymer is treated with the second, different reactive hydroxy—or amine group containing organic compound. This second different reactive compound can be selected from the various materials indicated above. Typical examples of preferred materials are siliconols, silanols, hydroxy-terminated polydialkylsiloxanes, dimethiconol, dimethicone (co)polyols, alkoxylated silanols etc., and mixtures of these compounds. Preferred second reactive hydroxy-group containing organic compounds are bis-hydroxypropoxyethyldimethicone and bis-hydroxyethoxypropyldimethicone. All or nearly all of the remaining unopened anhydride rings in the modified MA/O copolymer will be opened by the reaction with the second, different reactive organic compound and converted into functionalizing groups. By judicious choice of the amounts of reactants, temperature, catalyst and reaction time the degree of ring-opening can be controlled. In general from 95% to 10%, usually from 80% to 20%, preferably from 70% to 30% and particularly preferably 50% of the total number of originally present anhydride rings in the maleic anhydride/α-olefin copolymer will be opened by the second reactive organic compound. It is to be noted that if the first and/or the second functionalizing reactive agent is bi-functional, e.g. such as the above-mentioned dimethicone derivatives, some crosslinking may occur in the multiple functionalized MA/O copolymers.

If not all of the anhydride rings are opened with the above two types of functionalizing reactive agents, any remaining anhydride rings may, if desired, be opened by reacting them with a third reactive —OH or —NH₂ group containing organic compound, different from the one(s), used in the first and second step. A typical example of such a third reactive agent is ethanol.

Any free carboxy groups in the resulting finally obtained multiple functionalized maleic anhydride/α-olefin copolymer may remain unmodified, or they may be converted into salts such as the alkali metal, ammonium and substituted ammonium salts, or into esters or amides in manners, known per se. Preferably the carboxy groups remain free, in order to obtain optimum skin-substantivity of the multiple functionalized copolymers. The multiple functionalized copolymers of the invention will have a molecular weight of between 500 and 1,000,000, depending of course on the type of reactive agents used in their preparation.

The multiple functionalized copolymers of the present invention are useful for use in cosmetic compositions. They are skin-substantive, and can deliver particular cosmetic benefit agents and skin-actives to the skin. They can impart certain benefits to the compositions, e.g. pigment wetting, act as binders in compressed powder compositions, and particularly in solid and liquid compositions, they can act as improved film-forming agents and improved dispersants for pigments in a lipid phase. They can be used in mascara products, foundations, creams, lotions, sunscreen and suntan products, blush formulations, make-up products, eye shadows, and so. They are particularly useful in cosmetic products for the lips, such as lipsticks and fluid lip products for delivering an improved gloss and comfort and long-lasting wear properties without transferability. A multiple functionalized copolymer, prepared with a C₂₈-monoalcohol and a siliconol is particularly suitable for use in lipsticks, while a multiple functionalized copolymer, prepared with a C₁₄-monoalcohol and a siliconol is very suitable for use in fluid lip products.

The cosmetic compositions of the present invention comprise the multiple functionalized copolymer of the invention in an amount of 0.5-75%, preferably 0.75-35% and particularly preferably 1.0-20% by weight of the final composition.

The cosmetic compositions comprising the multiple functionalized copolymers can contain all the usual ingredients, commonly used in such compositions, such as resins, (co)polymers, silicones, volatile carriers, hydrocarbons, pigments, excipients such as talc, mica, silicas, titanium dioxide, zinc oxide, nylon particles, kaolin, calcium carbonate, starch, antioxidants, vegetable and mineral oils and fats, clays, conditioning agents, waxes, pearlescent agents, flavours, perfumes, vitamins, sunscreen agents, emollients, emulsifiers, skin care agents, surfactants, bactericides, preservatives and so on. With some of the above ingredients such as particulate excipients and pigments, the multiple functionalized copolymers of the present invention can be “linked”, thus increasing their wetting and dispersion in a lipid phase. The multiple functionalized copolymers of the invention are also quite suitable as coating agents for particulate materials such as powders, particulate pigments and other cosmetic ingredients in particle form.

All such suitable ingredients can be found in reference books such as the CTFA Cosmetic Ingredient Handbook, second Edition, The Cosmetic, Toiletries and Fragrance Association, Inc, 1988, 1992.

The invention will now further be illustrated by way of Examples. All percentages are by weight, unless otherwise indicated

### EXAMPLE 1

### Cross-Linked Silicone-Polyester C₁₄ Functionalized MA/O Copolymer.

| | 1A | 1B |
|---|---|---|
| | % | % |
| PHASE “A” | | |
| ISODODECANE | 64.10 | 16.10 |
| OCTADECENE/MA COPOLYMER | 16.30 | 39.10 |
| MYRISTYL ALCOHOL | 8.00 | 19.20 |
| PHASE “B” | | |
| WATER | 1.30 | 1.30 |
| CITRIC ACID | 0.30 | 0.30 |
| PHASE “C” | | |
| DIMETHYL SILOXANE, | 10.00 | 24.00 |
| HYDROXYALKYL-TERMINATED | | |

The copolymer was dissolved in isododecane at about 120° C. To the dispersion, myristyl alcohol was added and the mixture was allowed to react for 4 hours. To the resulting Phase A Phase B was then added and finally to complete the reaction hydroxyalkyl terminated dimethyl siloxane (Phase C) was added while heating for 1 hour at 110° C. The temperature was stabilized at 80° C. at which temperature the whole mass was kept for a further 12 hours. A dispersion of a silicone-C₁₄ polyester multiple functionalized MA/O copolymer in isododecane was obtained.

The degree of substitution in the multiple functionalized MA/O copolymer was in Phase A 79.9%, and in Phase C 19.2%, the total degree of substitution being 99.1%

### EXAMPLE 2

### Cross-Linked Silicone-Polyester C₁₆ Functionalized MA/O Copolymer.

| | % |
|---|---|
| PHASE “A” | |
| ISODODECANE | 38.40 |
| OCTADECENE/MA COPOLYMER | 16.30 |
| CETYL ALCOHOL | 6.50 |
| PHYTOSPHINGOSINE | 0.20 |
| PHASE “B” | |
| WATER | 1.30 |
| CITRIC ACID | 0.30 |
| PHASE “C” | |
| CYCLOPENTASILOXANE | 27.00 |
| DIMETHYL SILOXANE, | 10.00 |
| HYDROXYALKYL-TERMINATED | |

The copolymer was dissolved in isododecane at about 120° C. To the dispersion, cetyl alcohol and phytosphingosine were added and the mixture (Phase A) was allowed to react for 4 hours. Phase B was then added and finally to complete the reaction hydroxyalkyl terminated dimethyl siloxane was added while heating for 1 hour at 110° C. The temperature was stabilized at 80° C. at which temperature the whole mass was kept for a further 12 hours. A dispersion of a silicone-C₁₆ multiple functionalized polyester MA/O copolymer in isododecane and cyclopentasiloxane was obtained.

The degrees of substitution were in Phase A 58.8% and in Phase C 19.2%, the total degree of substitution being 78.0%.

### EXAMPLE 3

### Cross-Linked Silicone-Polyester C₁₈ Functionalized MA/O Copolymer

| | % |
|---|---|
| PHASE “A” | |
| ISODODECANE | 63.90 |
| OCTADECENE/MA COPOLYMER | 18.00 |
| DODECYLHEXADECANOL | 5.30 |
| PHYTOSPHINGOSINE | 0.20 |
| PHASE “B” | |
| WATER | 1.30 |
| CITRIC ACID | 0.30 |
| PHASE “C” | |
| DIMETHYL SILOXANE, | 11.00 |
| HYDROXYALKYL-TERMINATED | |

The copolymer was dissolved in isododecane at about 120° C. To the dispersion, dodecylhexadecanol and phytosphingosine were added and the mixture (Phase A) was allowed to react for 4 hours. Then Phase B was added and finally to complete the reaction hydroxyalkyl terminated dimethyl siloxane was added while heating for 1 hour at 110° C. The temperature was stabilized at 80° C. at which temperature the whole mass was kept for a further 12 hours. A dispersion of a silicone-C₁₈ polyester multiple functionalized MA/O copolymer in isododecane was obtained.

The degrees of substitution were in Phase A 23.2% and in Phase C 19.1%, the total degree of substitution being 42.3%.

### EXAMPLE 4

### Cross-Linked Silicone-Polyester C₁₄-C₁₈ Functionalized MA/O Copolymer

| | % |
|---|---|
| PHASE “A” | |
| ISODODECANE | 63.50 |
| OCTADECENE/MA COPOLYMER | 17.50 |
| HEXYLDECANOL | 4.80 |
| MYRISTYL ALCOHOL | 4.80 |
| PHYTOSPHINGOSINE | 0.30 |
| PHASE “B” | |
| WATER | 1.30 |
| CITRIC ACID | 0.30 |
| PHASE “C” | |
| DIMETHYL SILOXANE, | 7.50 |
| HYDROXYALKYL-TERMINATED | |

The copolymer was dissolved in isododecane at about 120° C. To the dispersion, hexyldecanol and phytosphingosine were added and the resulting mixture (Phase A) was allowed to react for 4 hours. Phase B was then added and finally to complete the reaction hydroxyalkyl terminated dimethyl siloxane was added while heating for 1 hour at 110° C. The temperature was stabilized at 80° C. at which temperature the whole mass was kept for a further 12 hours. A dispersion of a silicone-C₁₄-C₁₈ polyester multiple functionalized MA/O copolymer in isododecane was obtained.

The degrees of substitution were in Phase A 86.4% and in Phase C 13.4%, the total degree of substitution being 99.8%.

### EXAMPLE 5

### Beta-Sitosterol-Branched Polyester C₁₈-C₂₀ Functionalized MA/O Copolymer.

| | % |
|---|---|
| PHASE “A” | |
| TRIMETHYLOLPROPANE- | 76.00 |
| TRIISOSTEARATE | |
| OCTADECENE/MA COPOLYMER | 8.50 |
| BETA SITOSTEROL | 4.30 |
| PHASE “B” | |
| OCTYLDODECANOL | 11.20 |

The copolymer was dissolved in trimethylolpropane-triisostearate at about 120° C. To the dispersion, betasitosterol was added and the mixture (Phase A) was allowed to react for 4 hours. Phase B was then added to complete the reaction under heating for 1 hour at 110° C. The temperature was stabilized at 80° C. and the whole mass was kept at this temperature for a further 12 hours. A dispersion of a beta sitosterol-branched C₁₈-C₂₀ polyester multiple functionalized MA/O copolymer in trimethylolpropane-triisostearate was obtained.

The degrees of substitution were in Phase A 21.3% and in Phase B 77.2%, the total degree of substitution being 98.5%.

### EXAMPLE 6

### Cross-Linked Silicone-Beta Sitosterol Functionalized MA/O Copolymer

| | % |
|---|---|
| PHASE “A” | |
| ISODODECANE | 58.50 |
| OCTADECENE/MA COPOLYMER | 18.00 |
| BETA SITOSTEROL | 7.70 |
| PHASE “B” | |
| WATER | 1.30 |
| CITRIC ACID | 0.30 |
| PHASE “C” | |
| DIMETHYL SILOXANE, | 14.20 |
| HYDROXYALKYL-TERMINATED | |

The copolymer was dissolved in isododecane at about 120° C. To the dispersion, beta-sitosterol was added and the mixture (Phase A) was allowed to react for 4 hours. Phase B was then added and finally to complete the reaction hydroxyalkyl terminated dimethyl siloxane was added while heating for 1 hour at 110° C. The temperature was stabilized at 80° C. at which temperature the whole mass was kept for a further 12 hours. A dispersion of a silicone-beta sitosterol multiple functionalized MA/O copolymer in isododecane was obtained.

The degrees of substitution were in Phase A 36.1% and in Phase C 24.7%, the total degree of substitution being 60.8%.

### EXAMPLE 7

### Coating of Pigments with the Functionalized Copolymer of Example 5

| | |
|---|---|
| COMPONENTS | % |

PHASE “A”

| | |
|---|---|
| PIGMENTS (Talc-Mica-Iron oxide) | 82.00 |

PHASE “B”

| | |
|---|---|
| Functionalized copolymer of Example 5 | 8.00 |
| ISODODECANE | 10.00 |

Under strong turbulence Phase B was sprayed onto the pigments (Phase A). The solvent (isododecane) was then removed by drying the mixture in an oven at 80° C. for 12-24 hours.

### EXAMPLE 8

### Functionalized Copolymer (from Example 1) Containing Cosmetic Composition

(Lip Shine Fluid)

| COMPONENTS | % | % |
|---|---|---|
| Functionalized copolymer | 92.50 | 92.50 |
| | (1) | (2) |
| ETHYL ALCOHOL | 1.50 | |
| PIGMENTS | 5.00 | |
| WATER | 1.00 | |

(1) from Example 1A
(2) from Example 1B

### EXAMPLE 9

### Functionalized Copolymer (from Example 1) Containing Cosmetic Composition

(Lip Shine Stylo)

| COMPONENTS | % |
|---|---|
| CYCLOPENTASELOXANE | 32.00 |
| POLYETHYLENE | 7.00 |
| functionalized copolymer of Example 1 | 52.00 |
| ETHYL ALCOHOL | 2.00 |
| PIGMENTS | 5.00 |
| MICA | 2.00 |

### EXAMPLE 10

### Functionalized Copolymer (from Example 3) Containing Cosmetic Composition

(Mascara)

| | |
|---|---|
| COMPONENTS | % |

PHASE “A”

| | |
|---|---|
| WATER | 39.70 |
| GLYCERIN | 2.00 |
| HYDROXYETHYLCELLULOSE | 2.00 |
| ACACIA SENEGAL GUM | 6.00 |

PHASE “B”

| | |
|---|---|
| IRON OXIDE | 9.00 |

PHASE “C”

| | |
|---|---|
| HYDROGENATED RAPESEED OIL | 4.00 |
| GLYCERYLSTEARATE | 3.00 |
| CETEARYL GLUCOSIDE AND | 4.00 |
| CETEARYL ALCOHOL | |
| functionalized copolymer of Example 3 | 30.00 |

PHASE “D”

| | |
|---|---|
| PRESERVATIVE | 0.30 |

### EXAMPLE 11

### Functionalized Copolymer (from Example 5) Containing Cosmetic Composition

(Eye Shadow Compact Powder)

| COMPONENTS | % |
|---|---|
| TALC | 41.50 |
| MICA | 15.00 |
| IRON OXIDE | 25.00 |
| NYLON 12 | 6.00 |
| functionalized copolymer of Example 5 | 12.00 |
| PRESERVATIVES | 0.50 |

### EXAMPLE 12

### Functionalized Copolymer Coated Pigments (from Example 7) Containing Cosmetic Composition

(Eye Shadow)

| COMPONENTS | % |
|---|---|
| Coated pigments of Example 7 | 90.50 |
| NYLON 12 | 6.00 |
| functionalized copolymer of Example 5 | 3.00 |
| PRESERVATIVES | 0.50 |

1. A cosmetic composition comprising, in addition to conventional cosmetic ingredients, from 0.5-75% by weight of the composition of a multiple functionalized maleic anhydride/C₈-C₅₀ α-olefin copolymer, obtainable by reacting a maleic anhydride/C₈-C₅₀ α-olefin copolymer having a molecular weight of between 350 and 600,000 with a first reactive —OH or —NH₂ group containing organic functionalizing compound to open from 5% to 90% of the anhydride rings and reacting the resulting modified copolymer with a second reactive —OH or —NH₂ group containing organic functionalizing compound to open from 95% to 10% of the anhydride rings, said second reactive functionalizing compound being different from the first reactive functionalizing compound, said multiple functionalized copolymer having a molecular weight of between 500 and 1,000,000, wherein said first or said second reactive —OH group containing organic functionalizing compound is selected from the group consisting of straight chain alcohols with 8-30 carbon atoms, sitosterols and hydroxy terminated silicones.

2. The cosmetic composition according to claim 1, wherein any remaining anhydride rings in the multiple functionalized copolymer is opened by reaction with a third reactive —OH or —NH₂ group containing organic compound, said third reactive compound being different from the first and the second reactive —OH or —NH₂ group containing organic functionalizing compound.

3. The cosmetic composition according to claim 1 or 2, wherein the multiple functionalized copolymer contains an α-olefin containing from 10-24 carbon atoms.

4. The cosmetic composition according to claim 3, in which the α-olefin contains 18 carbon atoms.

5. (canceled)

6. The cosmetic composition according to claim 1, in which the alcohol is a straight chain monoalcohol with 14-28 carbon atoms

7.-8. (canceled)

9. The cosmetic composition according to claim 1, in which the second reactive organic functionalizing compound is a siliconol.

10. The cosmetic composition according to claim 9, in which the siliconol is bis-hydroxypropoxyethyldimethicone or bis-hydroxyethoxypropyldimethicone.

11.-14. (canceled)

15. A method for improving the gloss or long-lasting wear property of a lipstick or fluid lip product on the lip comprising applying to the lip a lipstick or fluid lip product comprising a cosmetic composition according to claim 1, claim 2, claim 3, claim 4, claim 6, claim 9 or claim 10.

16. A method of coating a particulate cosmetic ingredient comprising spraying a multiple functionalized copolymer according to claim 1, claim 2, claim 3, claim 4, claim 6, claim 9 or claim 10 onto the particulate cosmetic ingredients.
